(19) United States
(12) Patent Application Publication
(10) Pub. No.: US 2020/0168342 A1
(43) Pub. Date: May 28, 2020

(54) SYSTEMS AND METHODS FOR DRUG INTERACTION ALERTS

(71) Applicant: Humana Inc., Louisville, KY (US)

(72) Inventors: Ahmed Ghouri, San Diego, CA (US); Raghu Sugavanam, La Jolla, CA (US)

(21) Appl. No.: 16/775,438

(22) Filed: Jan. 29, 2020

Related U.S. Application Data
(63) Continuation of application No. 14/086,670, filed on Nov. 21, 2013, now abandoned.
(60) Provisional application No. 61/729,063, filed on Nov. 21, 2012.

(51) Int. Cl. G16H 70/00; G16H 50/30

(52) U.S. Cl. CPC G16H 70/00; G16H 50/30

(57) ABSTRACT

The present invention is a drug interaction alert system for alerting someone associated with a patient (including in one embodiment the patient himself) that a serious potential for drug interaction in health care has occurred for the patient. Health insurance claims data is used in the computerized process to evaluate the degree of seriousness of the drug interactions in care.

### CROSS REFERENCE TO RELATED APPLICATIONS

This application is a continuation of U.S. nonprovisional application Ser. No. 14/086,670 filed Nov. 21, 2013, which claims priority to U.S. provisional application Ser. No. 61/729,063 filed Nov. 21, 2012, the disclosures of each of which are hereby incorporated by reference in their entireties.

### FIELD OF THE INVENTION

The present invention relates to a system and method for analyzing drug interactions in patient care and promptly providing alerts to patients and/or care providers that a potentially serious drug interaction has arisen so that actions can be taken before a negative result occurs.

### BACKGROUND AND SUMMARY OF THE INVENTION

The health care profession has known that potentially dangerous drug interactions in patient care can lead to worsening health for the patient, and if that occurs there is the potential for much higher costs of health care for the patient. For example, it is known that the patient's health may deteriorate form a serious drug interaction. It is also known that a patient's health may be endangered from a serious drug interaction that could lead to hospitalization and expensive medical care that could have been avoided. Yet another example is in the area of pharmacies. Patients given multiple prescriptions that may be filled by different pharmacists (not knowing what another pharmacist has already filled for the same patient) may result in a serious drug interaction that could worsen a patient's condition.

All of the above scenarios are generally defined as drug interactions in care. There are many reasons why patients may have drug interactions in care, including but not limited to: 1) a contra-indicated drug prescribed for a given diagnosis; 2) Doctor A not knowing the drugs prescribed by Doctor B; and 3) patient's allergies to medications. Whatever the reason may be it is important to alert the patient or other entity concerned with the patient (such as their healthcare provider) of a possible drug interaction in care. Systems and methods have been used to alert various entities of medical information important to convey. For example, U.S. Pat. No. 5,754,111 shows one such system. Systems have been developed that use known medical data about a patient compared to historically collected medical data about drugs, conditions, interactions, contraindications, etc., to improve care for the patient. U.S. Pat. No. 7,809,585 describes such a system and method. The entireties of both of these patents are hereby incorporated by reference herein.

The danger in having a drug interaction is that it can result in undesirable outcomes for the patient. These undesirable outcomes may include a slower recovery, a complete lack of recovery potentially resulting in a chronic condition that could have been avoided, or in some cases, a dangerous worsening of a patient's condition. These results are potentially harmful to the patient's health and may also result in increased health care costs. The public, the news media, and some government agencies have become increasingly concerned about the cost and quality of health care. This exposure provides additional incentive for medical service and health insurance providers to work to control costs and improve patient care. Solutions that minimize the impact of drug interactions in care have the beneficial effects of improving the quality of care provided to patients, keeping patients healthy or improving their health, and reducing the cost of medical care.

Today's existing computer technologies allow the gathering and analysis of medical data of a patient's medical history. Today, a patient's medical records are often stored electronically, sometimes in records or files known as electronic medical records or EMRs. EMRs are well known such that details of their formation, updating, storing, sending, and receiving electronically via computer network are not explained herein. This electronic storage provides a repository of patient care information that can be leveraged by the health care system to improve a patient's care and reduce the overall cost of a patient's medical treatment. Methods of leveraging a patient's medical record(s) to reduce drug interactions in care have been developed but can fall short in their ability to reach patients in a timely way to avoid harmful drug interactions. Another problem is the so-called ‘crying wolf’ problem where too many warnings of possible drug interactions eventually begin to be ignored or just lost in so many warnings.

The present invention resides in improvements to the means for developing, generating, and communicating the existence of a drug interaction for a patient. This is done by scoring the level of seriousness of drug interactions on a patient by patient basis rather than on a universal basis and then promptly informing the patient of only the drug interactions that are scored as being serious for that patient. In this manner patients are more likely to take the actions required to eliminate the drug interaction. This communication is also delivered in a manner calculated to minimize the actual time of delivering the message to the patient. It is understood that once a drug interaction exists, a lengthy delay in communication can serve to aggravate or worsen the drug interaction and in doing so, to increase the likelihood of an unfavorable impact on the patient or an increase in the cost of care required to make the patient well.

Various known means of communication may be useful with the present invention. For example, outbound automated phone dialers in electronic communication with a computer system (such as used by telemarketing companies) may be useful in phoning someone about a drug interaction in care. Known computer automated email servers or texting services may also be used to contact someone about a drug interaction in care. Connected to a computer system that determines when to issue an alert or to send an alert, such known systems can be useful in delivering the alert.

In an exemplary embodiment of the present invention, a computer system analyzing patient medical data detects a drug interaction and initiates the generation of an alert message and delivery of a communication containing the message directly to the patient in the form of an automated phone call. A communication directly to the patient serves as an efficient and cost effective method to inform and encourage action to eliminate the drug interaction that exists. In another exemplary embodiment of the invention, such a phone call is made to a home health care service which then may attempt to contact the patient by an in-person visit to the patient's residence in order to check on the patient's condition and further encourage the patient to stop taking a particular drug. Other means for delivering the alerts will be described in the following detailed description, as will means for generating an alert.

In addition to the novel features and advantages mentioned above, other benefits will be readily apparent from the following descriptions of the drawings and exemplary embodiments.

### BRIEF DESCRIPTIONS OF THE DRAWINGS

While the appended claims set forth the features of the present invention with particularity, the invention and its advantages may be understood from the following detailed description taken in conjunction with the accompanying drawings, wherein identical parts are identified by identical reference numbers and wherein:

FIG. 1 shows a diagrammatic view of a computer network for use with the present invention;

FIG. 2 shows a diagrammatic view of delivery mechanisms for the alerts of the present invention;

FIG. 3 shows a flow chart of the algorithm of a preferred embodiment of the present invention.

### DETAILED DESCRIPTION OF THE INVENTION

The health care system comprises the interactions of three key entities; health care consumers (patients), health care providers, and health insurance providers. When a patient becomes ill, they visit their primary care physician if they have one. If not, a patient may visit an urgent care facility, emergency room, or as is becoming more common, a nurse practitioner that may have an office located in a grocery or drug store. The fact that a patient may visit any one of these health care providers creates the potential for a multiplicity of medical record locations. When one looks beyond the delivery of health care services to include the three key entities of the health care system, a common repository of patient health care exists.

When the patient visits a health care provider, they may receive a diagnosis, a recommended treatment protocol and possibly a prescription for medication. Depending on the disease or condition diagnosed, the health care provider may prescribe one or more medications. If the disease requires additional treatment, the treatment protocol may require follow-up care.

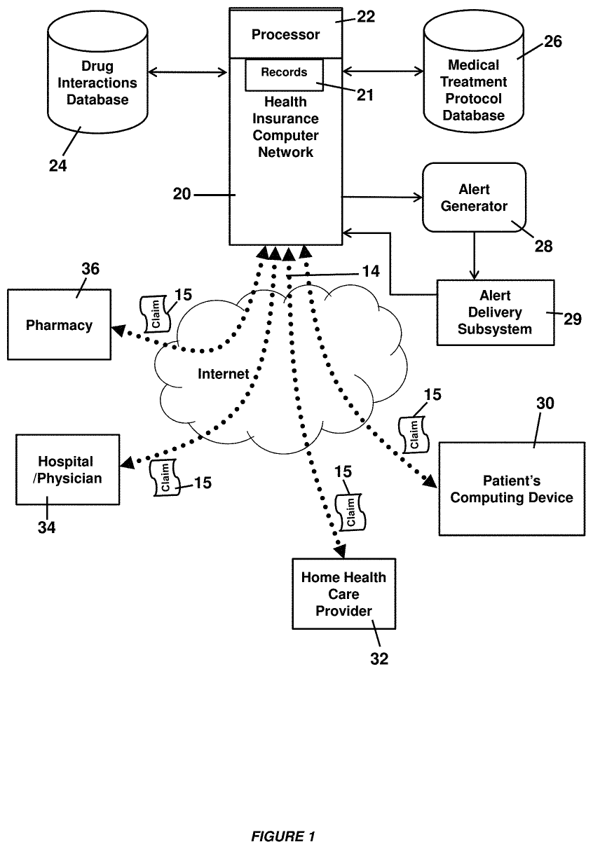

Referring now to FIG. 1, after visiting a health care provider 34, the provider and/or the patient 30 may submit a claim 15 for insurance payment or reimbursement to the patient's health insurance provider 20, via, for example, a computer network 14. That claim may contain details of the diagnosis and care provided, along with prescriptions written.

After meeting with the health care provider in a case where a prescription for a drug is written for the patient, the patient may fill a prescription at a pharmacy 36. The pharmacy and/or the patient may generate a claim 15 or report to the patient's health insurance provider 20. This claim may contain information including, for example, identifying information about the patient, the health care provider that generated the prescription, the drug prescribed, the quantity given, and the remaining refills available.

In addition to the patient's primary care physician there are other entities that may be providers of health care to a patient. These may include hospitals, medical specialists, physical therapists, home health care assistants 32, and many other types of care providers. When these additional health care providers perform a health care service for a patient, that provider and/or the patient may submit a claim to the patient's health insurance provider to obtain payment for the services provided. This insurance claim typically contains data sufficient to determine drugs prescribed and diagnosis.

A common theme in each of the previous scenarios is that claims from health care providers are submitted to the patient's health insurance provider. At the health insurance provider 20, the information is used to process 22 the patient's medical claims and provide payments to the respective service providers according to the terms of the agreement between the patient and the health insurance provider. This information allows the health insurance provider to determine what benefits the patient is entitled to and whether the health care provider is following generally accepted methods of treatment for particular medical conditions.

When a claim is submitted, the patient's claims become part of his or her health record 21 maintained by the health insurance provider on its computer network 20. Because this health record 21 persists after a patient's claims have been paid, the health record serves to gather and maintain information taken from claims submitted by a patient while he or she is served by the health insurance provider. The result is an accumulation of data gathered over time concerning the patient which may include among other data, past illnesses or conditions, drugs prescribed, harmful drug reaction, allergies, and family medical history as provided by the patient.

The sheer volume of data and number of patients supported by a health insurance provider requires that in order to perform even the basic function of processing and paying claims that the data be managed by a sophisticated computer system 22 containing one or more processors. Exposure to a computer system allows the data to be subject to various forms of analysis. One such type of analysis serves as a means to detect drug interactions in care.

In some cases, drug interaction may result in a patient not recovering as quickly, suffering from additional illness or disease, or, developing potentially life-threatening complications. For the patient, health care providers, and society, a continued or worsening illness results in frustration, loss of enjoyment of life, potential injury, and when taken in the aggregate, an impact on the productivity of the economy as a whole. A follow-up visit to a primary physician, the resulting prescription cost, and follow-up care costs are just a fraction of what a prolonged recovery or more serious illness may cost in terms of an extended hospital stay that may result if the patient is not informed of a potentially harmful drug interaction. There is a need for means for prompting the patient about a potentially harmful drug interaction to avoid a situation in which a relatively manageable medical event progresses to something much more severe, and more costly to treat.

There are many reasons why a patient may experience a drug interaction. A patient may not inform one health care provider that he is being seen by a second health care provider. A patient may forget to mention to a health care provider that he is already taking medication, or that he suffers from a medical condition or disease. A drug interaction system of the present invention provides the information needed to communicate the potentially harmful interaction.

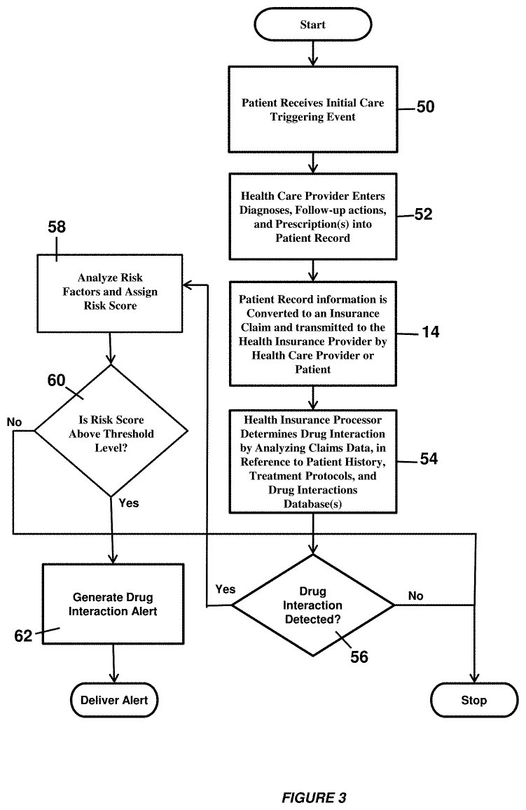

With reference to FIG. 3, a drug interaction system may be implemented by analyzing a patient's medical treatment history. The system preferably looks for drugs prescribed or drug prescriptions filled, and compares that to a list for the patient of either: a) other drugs presently being taken; or b) present medical conditions. Using interaction tools 24, 26 the computer system identifies potentially harmful drug interactions.

For the purposes of drug interaction analysis and alerts, a health insurance provider is in a unique position. Unlike any single health service provider or primary care physician, the health insurance provider has access to an aggregate of the patient's medical history across practically all forms of health care and sources of that care. For example, a primary care physician may not have a complete record of a patient's care if that patient were to see a health care provider while on vacation in another city or state and that second health care provider failed to deliver a record of the visit or diagnosis to the patient's primary care physician. Another example might be a situation in which a patient provided the incorrect or incomplete name of his primary care physician when visiting an emergency room. Because a patient and/or care provider will very likely seek insurance coverage in every instance of care, the health insurance provider may have the most complete record of a patient's history.

To implement a computerized drug interaction detection system of the present invention, claims data contained in the record for a particular patient may be analyzed with regard to the type of treatment received, the disease or condition diagnosed, prescription(s) written, and/or the treatment protocol recommended to the patient. A computer algorithm may first check the patient record for any prescribed drugs for which claims were submitted. This may result in the detection of a record detailing prescription medication and diagnosis by the health care provider. An analysis is then conducted during which this claims data is compared to a set of protocols of predetermined and generally accepted medical standard determinations of ‘drug on drug’ interactions and/or ‘drug on disease’ interaction. Such protocols may be acquired from a recognized source(s) of such data or may be derived internally by a health insurance provider reviewing historical outcomes data for its insured patients over a lengthy period of time. Discrepancies between the results of these analyses may be reviewed by an exception process.

When a triggering event 50, (e.g., filling a prescription 52) occurs for a patient, the processor 22 as shown in FIG. 1 performs algorithmic computation based on then-current data, including that data specific to the patient, the patient's disease, ailment, or condition, and the prescribed drug or drugs, and the protocol is applied to the patient's care. Using database(s) 54 of patient specific data, and drug on drug interaction data, and/or drug on disease or condition data, the system first acts to identify the potential for a problematic drug interaction 56 due to the use of a particular drug by the patient. For example, the system of the present invention recognizes drug allergies specific to a patient, and the system recognizes diseases or medical conditions specific to a patient. Once the system identifies a potential for a drug interaction in a particular patient the invention identifies 58 the likely degree of danger or problems resulting from that patient taking that drug(s). For example, while a particular drug may be known to cause problems in people having hypertension if a particular patient suffering from hypertension has not had a prior problem with use of that drug then the likelihood of a serious problem is less for that patient. Next, the invention goes on to score 58 the relative danger of use of a particular drug by a particular patient, for example, on a scoring format of 0-100, with zero indicating no potential for a drug interaction and 100 indicating extreme danger and a practically certain drug interaction with very serious negative health results for the patient. For example, the user of the present invention may set the system to establish a score alert value 60 over which alerts will be generated 62 and sent, and below which alerts will not be sent. For example, a user may set the score alert value at “70” so that only drug interaction scores above “70” would receive an alert message. Those scoring “70” or under “70” may be monitored by the system of the present invention for further developments but not alerted. Various factors can be assigned various weight in this scoring analysis, using the processor(s) 22 and the record 17 for the patient, along with database(s) 24, 26. For example, factors such as a patient's body weight, age, allergies, existing medical condition(s), and medical history may be considered and weighted as more important or less important considerations in developing a score for use of a particular drug by that patient. A drug allergy for that patient would be weighted of high importance. A patient's age or sex may be weighted with relative less importance. Consistently, historically reported dangerous drug interactions for a particular disease or medical condition would be weighted with higher importance.

Once all selected factors are weighted and analyzed, the processor analyzes the drug(s) prescribed to the patient under two queries: is a drug prescribed likely to have a serious unhealthy interaction in this patient based on another drug the patient is also taking or has also been prescribed to take; and, is a drug prescribed likely to have a serious unhealthy interaction in this patient based on the patient's diagnosed condition (e.g., drug allergy) or disease. The data is processed and a risk score is assigned to a prescribed drug for that patient. In an exemplary embodiment the total risk score is automatically calculated by the system adding up points associated with each weighted risk factor found to be relevant to the patient and the drug prescribed. If the total risk score is below a threshold value of risk then no alert is generated. If the risk score is above the threshold value of risk an alert is generated and sent to the patient or someone connected to the patient, warning the receiver of the dangers of continued use of the prescribed drug.

Once a threshold care/triggering event has been determined, the algorithm is then implemented to monitor patient data in the form of other claims submitted for drug prescriptions and/or care services. For example, a triggering event occurs for a patient (such as diagnosis of pneumonia and prescribed antibiotic) on a particular date. An insurance claim is filed for the patient and the health insurance company enters the data from the claim into a computer network. The drug interaction detection algorithm of the present invention stores the triggering event in a file associated with the patient's id, for future reference. Next, the present invention preferably automatically analyzes the diagnosis and drug prescribed to see if the drug is contra-indicated for the patient or for the diagnosis. Based on a look-up table of particular health care triggering events (e.g., diagnosis of pneumonia) (that may be performed by processor 22) and generally recognized recommended treatment protocols 26 (e.g., treat with particular drugs), known drug interactions in database 24, and any patient specific data such as allergies to particular drugs, a first drug interaction analysis is performed. The present invention then stores the patient's claims data in personalized records 21 in the computer network associated with the patient's id. In a preferred embodiment the present invention performs a follow-up analysis when an additional drug is prescribed for the patient, and/or when an additional diagnosis is made of the patient's condition. If a potentially harmful drug interaction is identified, the computer generates an alert using an alert generator 28 which may be delivered via an alert delivery subsystem 29 of the present invention.

The alert delivery subsystem 29 may be configured to deliver the alerts through the health insurance computer network 20 or an electronic messaging module 13 included in the subsystem 29, to patient devices (e.g., phones, mobile computing devices, home computers, etc.) or to other entities on behalf of the patient (e.g., a health care provider device). The delivery subsystem may use various forms of communications to deliver the alerts (e.g., voice message, text message, email message, warning message to an EMR, etc.). The alert message itself may take many forms, including but not limited to: a simple direct informational message that a particular patient may experience a harmful drug interaction and warning the patient or health care provider that the patient should consult his health care provider soon to seek medical advice concerning the potential interaction; a more robust message describing the particular drug interaction and the potential dangers of not accomplishing a follow-up consultation; or a very short message that the patient is requested to contact their health care provider or health insurance company representative about an important message for them.

Two examples may be helpful to illustrate the process. In a first example, a claim is submitted that details a diagnosis and prescribed drug by a diagnosing physician. The existence of a first prescribed drug triggers the drug interaction detection algorithm. The algorithm then monitors for claims submitted by or on behalf of the patient for a subsequent second prescribed drug for the patient. The algorithm also performs analysis of the prescribed drug(s) in view of the patient's diagnosis, any pre-existing condition(s), and other available patient data. In this manner, drug on drug interaction(s) and drug on disease/condition interaction(s) are detected. The indication that the patient may experience a drug interaction, is the result of a claim(s) to the health insurance provider.

The drug interaction detection algorithm therefore consists of a means for analyzing patient claims data to detect claims that contain prescribed treatment protocols (sparking a triggering event). When such a claim is detected for a patient, the algorithm stores a record of it and initiates a first drug interaction look-up based on the drug and diagnosis associated with the prescribed treatment protocol for a given patient. The algorithm then monitors subsequent ones of that patient's claims submitted for indications that additional drug(s) were prescribed, for follow-up analysis. If the algorithm determines that a potentially harmful drug interaction may occur in the patient, an alert is generated for further action.

The drug interaction detection algorithm and analysis process for monitoring claims data are preferably performed on a processor system 22 maintained by the health insurance provider. This processor system is connected via a network 20 that houses patient claim data. Various other computer systems are able to access the network including a system for entering claims data into the network. This claim entry system may be implemented in a number of traditional ways.

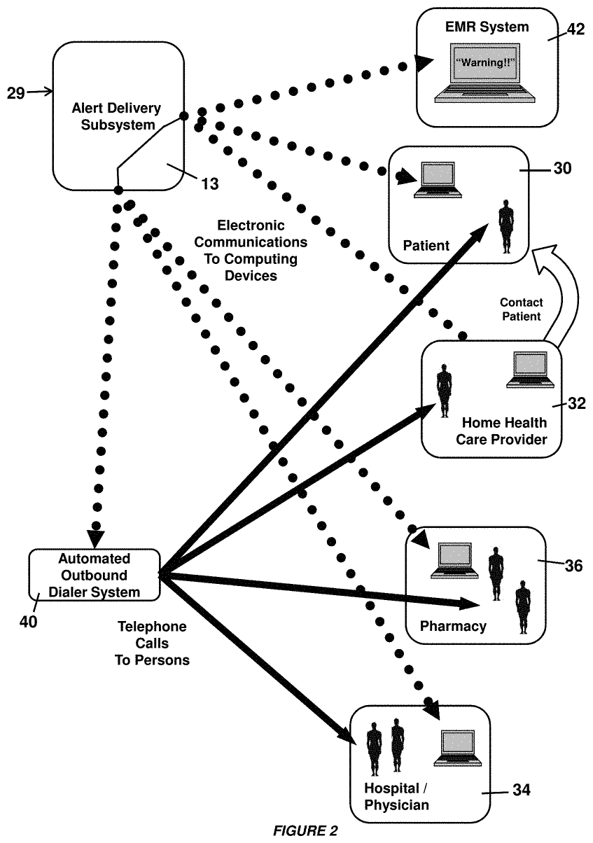

Once the drug interaction algorithm identifies a potential drug interaction for a patient, the algorithm triggers an alert. This alert may trigger a further review process at the health insurance provider or alternatively at the health care provider associated with the claim. Once any review is completed the drug interaction alert may be communicated to an individual or organization by an alert delivery subsystem 29 as shown in another embodiment of the invention in FIG. 2, to follow up with the health care provider or patient. An improvement in drug interaction detection comprises a computerized notification system which may be maintained in association with the health insurance provider and connected to the computer network and database which contains patient records and the results of the drug interaction detection algorithm's analysis. This notification system may be connected to an automated telephone system 40 which when activated by the subsystem 29 generates phone calls to notify patients that a potential drug interaction has been detected. This notification system may also be connected to a computerized messaging system capable of sending emails, text messages, or other means of electronic messaging that a person skilled in the art will realize additional possibilities for messaging.

Again, in FIGS. 1 and 2 there is shown the computerized system of the present invention. A health insurance computer system 20 may be in electronic communication via the Internet with various other computer systems, including, but not limited to, a pharmacy computer system 36, a hospital computer system 34, a patient personal computer system 30, or other health care provider computer system 32.

With reference to FIG. 3 there is shown a flow diagram of the computer program algorithm, of the present invention. Health insurance claims data is received by the health insurance computer system and the program identifies recent medical care provided for a particular patient. Next, the program preferably consults medical treatment protocol database 26 to determine the generally recognized steps in the treatment of the patient's condition, disease, ailment, or surgery. Alternative sources may be used for the database 26. Commercially available treatment protocols may be used, or the healthcare provider's treatment protocol may be used, or an in-house generated treatment protocol may be used. Insurance companies are particularly well situated with years of patient healthcare data to know treatment protocols that work well and ones that don't. Once an alert is generated the system invokes the alert delivery subsystem to get the alert to its proper destination.

With reference to FIG. 2 the alert, once generated by an alert delivery subsystem 29, may be delivered over various channels to predetermined destinations. For example the alert may be sent automatically through an email server to a destination email server for alerting a predetermined party of the drug interaction via an email message. The alert may be delivered to an electronic medical record (EMR) 42 housed at a health care provider computer system and presented as a written warning near the top of the EMR to be seen by health care professionals and/or a patient. Many methods of delivery of the alert are contemplated by the present invention.

The data used by the health insurance company computer system to determine a drug interaction alert may be derived from health insurance claims data, or various other sources, including health care provider data received from heath care provider data systems, patient entered data received directly from the patients/insureds, or from practically any other medical data source, including pharmacies, home health care facilities, etc. The actual alerts generated by the system of the present invention may be in the form of printed words on paper, electronic words in electronic environment such as electronic messages or texts, voice message, or even speech provided from a delivery person (alerted by the system of the present invention) direct to the patient or care provider.

A triggering event which starts the process of the present invention may include but is not limited to, surgery, a date of diagnosis of a disease, a date of treatment beginning, a date an initial prescription for medication is filled, a date of a doctor's office visit by a patient, or practically any other identifiable date from which a subsequent follow up activity should occur. By identifying the specific nature of the triggering event the processor accesses a database of medical treatment protocols 16 or subsequent drug(s) prescribed, to determine any drug interaction(s). Once the system identifies the drug interaction an alert signal is actuated automatically by the system and the delivery of the alert is handled by the alert delivery subsystem of the present invention.

An example of the present invention in use is helpful to explain its benefits and features. A patient is away from her home location and is seen by an urgent care physician and is diagnosed with a urinary tract infection. An antibiotic is prescribed for the patient and she fills the prescription at a pharmacy soon thereafter. The patient forgets to inform the urgent care physician that she is already taking another medication for acne. At each place of care, the urgent care facility and the pharmacy, the patient presents her health insurance card for payment for the services rendered. Both care facilities enter this health insurance information into their computer systems and send it to the health insurance company computer network electronically for payment. Included in the data the urgent care facility supplies, is patient id information, diagnosis code for urinary tract infection, and treatment code for the prescribed treatment, cost and payment data, and perhaps other data. Included in the data the pharmacy supplies is the patient id information, the code for the drug dispensed, the amount of the drug dispensed, the date the drug was dispensed to the patient, and cost and payment data, plus other data.

Now the health insurance computer network has sufficient information to prompt a triggering event for the patient, which begins the process of drug interaction analysis. A computer processor associated with the health insurance company computer network first analyzes the claims data to see if the prescribed drug is indicated and likely to be beneficial for a urinary tract infection. The health insurance company network has this available information stored in a database from years of historical data collected for insured patients who have experienced urinary tract infections and have been treated with various medications successfully or unsuccessfully. If the health insurance company computer network determines that the drug prescribed is not indicated or not likely to be beneficial for the patient an alert is automatically generated and sent to the care physician and/or patient via email or automated phone call or sent to an EMR. In this example the alert is a warning message that the drug prescribed is not indicated for this ailment and the patient should be seen again by the physician.

Next, continuing with the above example, the health insurance computer network detects that the patient has in prior weeks filled a prescription for acne medication (that also had an insurance claim from which this data was collected). The computer system checks a look-up table in a database to see whether the drug prescribed for the urinary tract infection is likely to have a harmful impact on the patient when taken with the drug for acne. If yes, the computer system generates an alert and sends it automatically and electronically to the pharmacy and/or the patient and/or the physician to have them contact the patient to stop the use of one of the medications until a consultation with a physician has occurred.

The present invention has been described herein with reference to the figures and various preferred embodiments, but is not to be construed as limited thereto. The invention is susceptible to modifications and variations that fall within the following claims. The claims of the present invention are not limited to the embodiments described in detail herein but are intended to have broad scope to capture the full scope of the present invention as allowed by law.

What is claimed is:

1. A method for preemptively alerting users to potentially harmful drug interactions, said method comprising the steps of:
providing a first database within a computer network, said first database comprising historical health data for multiple healthcare visits for a patient, wherein said historical health data comprises identification of at least one medication that has been prescribed for or dispensed for the patient, and wherein said historical health data is associated with at least two different healthcare providers;
receiving, at the computer network, new healthcare data associated with the patient, wherein said new healthcare data comprises an indication of a newly diagnosed disease;
associating, electronically and automatically, by utilization of a medical treatment protocol process associated with the computer network, the newly diagnosed disease with at least one drug known to be previously prescribed to other patients to treat said disease;
determining, electronically and automatically, by way of drug interaction data provided at a drug interaction database associated with the computer network, that taking the at least one drug previously prescribed to other patients to treat said disease in combination with said at least one medication that has been prescribed for or dispensed for the patient can result in a potentially harmful interaction; and
generating, electronically and automatically, an alert notice to either the patient or a healthcare professional for said patient, of said potentially harmful interaction.

2. The method of claim 1 further comprising the steps of:
assigning, electronically and automatically, at the computer network, an indication of a degree of likely severity to the potentially harmful interaction, and including the degree of likely severity in said alert notice.

3. The method of claim 2 wherein:
said indication of severity is selected based upon factors comprising the patient's body weight, age, any allergies, and any pre-existing medical conditions.

4. The method of claim 3 wherein:
the step of assigning the indication of severity comprises the sub-step of assigning a weighting to each of the factors.

5. The method of claim 4 wherein:
a lower weighting is assigned to the body weight and the age of the patient relative to the weighting assigned to the allergies, and pre-existing medical conditions.

6. The method of claim 2 wherein:
said indication of severity comprises a number or score between zero and 100.

7. The method of claim 1 wherein:
said alert notice is delivered as at least one of the group consisting of: an automated call, a text message, an email, a printed document, and an electronic medical record system notification.

8. A system for preemptively alerting users to potentially harmful drug interactions resulting from healthcare visits with different healthcare providers, said system comprising:
a health insurance computer network comprising one or more electronic storage devices, one or more processors, and at least one database comprising a plurality of health insurance claims for a plurality of insured individuals, wherein said health insurance computer network is associated with a plurality of pharmacies, hospitals, healthcare provider practices, and insured computing devices by way of the internet for receiving said plurality of health insurance claims, and wherein said plurality of health insurance claims comprise a new claim and a number of historical claims for a particular one of the plurality of insured individuals, wherein said new claim comprises at least one code indicating diagnosis of at least one new disease, wherein said at least some of said number of historical claims comprise prescribed drugs and at least some other of said number of historical claims comprise diagnosed diseases, and wherein each of said number of historical claims are associated with one of at least two different healthcare providers;
a drug interaction database associated with said health insurance computer network and comprising data regarding combinations of drugs with other drugs and combinations of drugs with diseases known to cause potentially harmful interactions;
a medical treatment protocol database associated with said health insurance computer network and comprising a number of codes, including the at least one code, each associated with one or more drugs, wherein each of the number of codes corresponds with an associated disease, and wherein the one or more drugs associated with a given one of the number of codes comprise those which are commonly prescribed to treat the associated disease;
an alert generation module associated with said health insurance computer network and configured to generate alert messages;
an alert delivery subsystem associated with said health insurance computer network and configured to deliver alert messages; and
software instructions, stored at said one or more electronic storage devices, which when executed by said one or more processors configures said one or more processors to:
receive notification of a triggering event;
query the at least one database with identification information for the particular one of the plurality of insured to retrieve the one or more prescribed drugs from said historical claims and the one or more diagnosed diseases from said historical claims associated with the particular one of the plurality of insured;
query the medical treatment protocol database with the code to retrieve the one or more drugs associated with the code;
query the drug interaction database to determine if any of the retrieved drugs from the medical treatment protocol database are known to cause potentially harmful interactions when taken in combination with any of the retrieved prescribed drugs from said historical claims or when having any of the retrieved diagnosed diseases from said historical claims;
assign a severity score for each of retrieved drugs from the medical treatment protocol database known to cause potentially harmful interactions;
direct the alert generation module to generate an alert identifying each of the retrieved drugs from the medical treatment protocol database determined to cause potentially harmful interactions and the associated severity score; and
direct the alert delivery subsystem to deliver the generated alert to each of the users, wherein said users comprise a healthcare provider associated with the new claim.

9. The system of claim 8 wherein:
said alert is delivered to the healthcare provider by way of a notification at an electronic medical record system associated with the healthcare provider.

10. The system of claim 9 wherein:
said one or more users comprise all pharmacies, hospitals, and healthcare provider practices from which said health insurance claims associated with said particular one of the plurality of insured individuals were received at said database.

11. The system of 10 wherein:
said one or more users comprise all insured computing devices from which said health insurance claims associated with said particular one of the plurality of insured individuals were received at said database.

12. The system of claim 8 further comprising:
additional software instructions, stored at said one or more electronic storage devices, which when executed by said one or more processors configures said one or more processors to query the drug interaction database to determine if any of the newly diagnosed diseases are known to cause potentially harmful interactions in combination with any of the one or more prescribed drugs from said historical claims, and assign a severity score for each of newly diagnosed diseases known to cause potentially harmful interactions, wherein said alert comprises each of newly diagnosed diseases known to cause potentially harmful interactions and the associated severity score.

13. The system of claim 8 wherein:
said triggering event comprises receipt of the new claim.

14. A method for preemptively alerting users to potentially harmful drug interactions resulting from healthcare visits with different healthcare providers, said method comprising the steps of:
receiving, at a records database of a health insurance computer network associated with an insurance provider, a new health insurance claim comprising a diagnosis code and a treatment code;
querying a medical treatment protocol database associated with the health insurance computer network with the diagnosis code and the treatment code to retrieve at least one drug, wherein the retrieved at least one drug is commonly prescribed to treat a disease associated with the diagnosis code and is associated with the treatment code;
querying the records database to generate a list of drugs previously prescribed to said insured based on historical health insurance claims for said insured over a predetermined lookback period and a list of diseases previously diagnosed for said insured based on said historical health insurance claims for said insured over the predetermined lookback period;
determining, utilizing a drug interaction database associated with the health insurance computer network, that taking one or more of the retrieved at least one drug and at least one of the list of drugs or that taking one or more of retrieved at least one drug when having at least one of the list of diseases could result in one or more potentially harmful interactions;
assigning, at the health insurance computer network, a severity score to each of the one or more potentially harmful interactions, wherein each severity score is determined using factors comprising a body weight, an age, one or more diagnosed allergies, one or more existing medical conditions, and one or more medical history data points for the insured, and wherein a weight is assigned to each of said factors;
generating an alert comprising each of the retrieved at least one drug associated with each of the one or more potentially harmful interactions and the associated severity score for each of the one or more potentially harmful interactions;
determining that one or more of the severity scores is above a predetermined threshold;
providing, by way of automated calls, text messages, emails, or electronic notifications, messages comprising the alert to contact information associated with the insured, the healthcare provider, and pharmacies associated with said insured as determined from said historical health insurance claims for said insured over the predetermined lookback period.

15. The method of claim 14 wherein:
a lower weighting is assigned to the body weight and the age of the insured relative to the weighting assigned to the one or more diagnosed allergies, one or more existing medical conditions, and one or more medical history data points for the insured.
